(19) United States
(12) Patent Application Publication
(10) Pub. No.: US 2024/0359009 A1
(43) Pub. Date: Oct. 31, 2024

(54) Systems and Methods for Calibration of Retinal Prosthetics

(71) Applicant: The Board of Trustees of the Leland Stanford Junior University, Stanford, CA (US)

(72) Inventors: Eduardo Jose Chichilnisky, Stanford, CA (US); Praful K. Vasireddy, Stanford, CA (US); Nishal Pradeepkumar Shah, Fremont, CA (US)

(73) Assignee: The Board of Trustees of the Leland Stanford Junior University, Stanford, CA (US)

(21) Appl. No.: 18/646,450

(22) Filed: Apr. 25, 2024

Related U.S. Application Data
(60) Provisional application No. 63/498,204, filed on Apr. 25, 2023.

(51) Int. Cl. A61N 1/36; A61N 1/05

(52) U.S. Cl. CPC A61N 1/36046; A61N 1/0543

(57) ABSTRACT

Systems and methods for calibration of retinal prosthetics in accordance with embodiments of the invention are illustrated. A closed loop calibration process is described whereby a multi-electrode stimulation regime can be calibrated to a given user's retina. Multi-electrode stimulation can provide increased stimulation selectivity, but significantly increases complexity. Systems and methods described herein provide computational steps that significantly reduce the amount of trials and computation required in order to achieve clinically viable selectivity, making closed-loop calibration of retinal prosthetics possible in significantly less time than open-loop calibration.

### CROSS-REFERENCE TO RELATED APPLICATIONS

The current application claims the benefit of and priority under 35 U.S.C. § 119(e) to U.S. Provisional Patent Application No. 63/498,204 entitled “Systems and Methods for Calibration of Retinal Prosthetics” filed Apr. 25, 2023. The disclosure of U.S. Provisional Patent Application No. 63/498,204 is hereby incorporated by reference in its entirety for all purposes.

### GOVERNMENT LICENSE RIGHTS

This invention was made with Government support under contract 1828993 awarded by the National Science Foundation and under contracts EY021271 and EY032900 awarded by the National Institutes of Health. The Government has certain rights in the invention.

### FIELD OF THE INVENTION

The present invention generally to the calibration of retinal prosthetics, namely closed-loop capable calibration methods using parametric modeling.

### BACKGROUND

The human eye is the organ responsible for sight. The eye is an optical device made up of several layers. The innermost, light-sensitive layer of the eye is the retina. The retina consists of several layers of photoreceptors and neurons that receive light and transmit visual information to the brain. Damage to the retina can result in loss or reduction in vision. Two common eye diseases, retinitis pigmentosa and age-related macular degeneration, cause many cells in the retina to die. Retinal prosthetics (also sometimes referred to as “Artificial Sight Prosthetics”, “Epiretinal Implants”, or “Bionic Eyes” are a class of medical devices designed to interface with the neurons in the retina in order to restore vision. Retinal prosthetics are described in detail in U.S. patent application Ser. No. 17/441,261 titled “Systems and Methods for Artificial Sight Prosthetics”, filed Mar. 23, 2020, the disclosure of which is hereby incorporated by reference in its entirety.

### SUMMARY OF THE INVENTION

Systems and methods for calibration of retinal prosthetics in accordance with embodiments of the invention are illustrated. One embodiment includes a method of calibrating a retinal prosthetic, including: instantiating a parametric model for multi-electrode stimulation, stimulating a plurality of retinal ganglion cells (RGCs) using a set of adjacent electrodes in a microelectrode array, recording activation responses of the plurality of RGCs in response to the stimulation, fitting the parametric model to the recorded activation responses, determining a plurality of stimulation patterns for a next testing iteration using sequential measurement optimization, where each stimulation pattern comprises a current level and a voltage for each electrode in the set of adjacent microelectrodes, stimulating the plurality of RGCs using a sampling of the determined plurality of stimulation patterns, stimulating the plurality of RCGs using a different set of stimulation patterns, recording new activation responses of the plurality of RGCs in response to the sampled stimulation patterns and the different set of stimulation patterns, fitting the parametric model to the new activation responses, and selecting a stimulation pattern that is most selective for a target RGC in the plurality of RGCs using the parametric model.

In another embodiment, the sequential measurement optimization is A-optimal design.

In a further embodiment, recording activation responses includes determining locations and an electrical image of each RGC in the plurality of RGCs.

In still another embodiment, the parametric model is pᵢ(x)=σ(w_{i,0}+wᵢ^{T}x), where wᵢ∈ [symbol] ^{d} is a vector of weights on multi-electrode currents, w_{i,0} is a scalar bias term for site i, and σ(x)=1/(1+exp (−x)).

In a still further embodiment, a cardinality of the set of different stimulation patterns is determined by optimizing

$${T}^{*} = {arg min}_{T > 0} [ tr ( var ( p ( \hat{w} ) ) ) + λ \vert {‖ T ‖}_{1} - {T}_{budget} \vert ] ,$$

where T_{budget} is the cardinality, and λ is an /1-regularization parameter.

In yet another embodiment, a retinal prosthetic includes a microelectrode array comprising a plurality of electrodes, a processor, and a memory, the memory containing a calibration application that configures the processor to: instantiate a parametric model for multi-electrode stimulation, stimulate a plurality of retinal ganglion cells (RGCs) using a set of adjacent electrodes in a microelectrode array, record activation responses of the plurality of RGCs in response to the stimulation, fit the parametric model to the recorded activation responses, determine a plurality of stimulation patterns for a next testing iteration using sequential measurement optimization, where each stimulation pattern comprises a current level and a voltage for each electrode in the set of adjacent microelectrodes, stimulate the plurality of RGCs using a sampling of the determined plurality of stimulation patterns, stimulate the plurality of RCGs using a different set of stimulation patterns, record new activation responses of the plurality of RGCs in response to the sampled stimulation patterns and the different set of stimulation patterns, fit the parametric model to the new activation responses, and select a stimulation pattern that is most selective for a target RGC in the plurality of RGCs using the parametric model.

In a yet further embodiment, the sequential measurement optimization is A-optimal design.

In another additional embodiment, to record activation responses, the calibration application further configures the processor to determine locations and an electrical image of each RGC in the plurality of RGCs.

In a further additional embodiment, the parametric model is pᵢ(x)=σ(w_{i,0}+wᵢ^{T}x), where wᵢ∈ [symbol] ^{d} is a vector of weights on multi-electrode currents, w_{i,0} is a scalar bias term for site i, and σ(x)=1/(1+exp (−x)).

In another embodiment again, the calibration application further configures the processor to determine a cardinality of the set of different stimulation by optimizing

$${T}^{*} = {arg min}_{T > 0} [ tr ( var ( p ( \hat{w} ) ) ) + λ \vert {‖ T ‖}_{1} - {T}_{budget} \vert ] ,$$

where T_{budget} is the cardinality, and λ is an /1-regularization parameter.

In a further embodiment again, non-transitory machine readable medium containing program instructions that are executable by a set of one or more processors to perform a method including instantiating a parametric model for multi-electrode stimulation, stimulating a plurality of retinal ganglion cells (RGCs) using a set of adjacent electrodes in a microelectrode array, recording activation responses of the plurality of RGCs in response to the stimulation, fitting the parametric model to the recorded activation responses, determining a plurality of stimulation patterns for a next testing iteration using sequential measurement optimization, where each stimulation pattern comprises a current level and a voltage for each electrode in the set of adjacent microelectrodes, stimulating the plurality of RGCs using a sampling of the determined plurality of stimulation patterns, stimulating the plurality of RCGs using a different set of stimulation patterns, recording new activation responses of the plurality of RGCs in response to the sampled stimulation patterns and the different set of stimulation patterns, fitting the parametric model to the new activation responses, and selecting a stimulation pattern that is most selective for a target RGC in the plurality of RGCs using the parametric model.

In still yet another embodiment, the sequential measurement optimization is A-optimal design.

In a still yet further embodiment, recording activation responses comprises determining locations and an electrical image of each RGC in the plurality of RGCs.

In still another additional embodiment, the parametric model is pᵢ(x)=σ(w_{i,0}+wᵢ^{T}x), where wᵢ∈ [symbol] ^{d} is a vector of weights on multi-electrode currents, w_{i,0} is a scalar bias term for site i, and σ(x)=1/(1+exp (−x)).

In a still further additional embodiment, a cardinality of the set of different stimulation patterns is determined by optimizing

$${T}^{*} = {arg min}_{T > 0} [ tr ( var ( p ( \hat{w} ) ) ) + λ \vert {‖ T ‖}_{1} - {T}_{budget} \vert ] ,$$

where T_{budget} is the cardinality, and λ is an /1-regularization parameter.

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### SUMMARY OF THE INVENTION

Additional embodiments and features are set forth in part in the description that follows, and in part will become apparent to those skilled in the art upon examination of the specification or may be learned by the practice of the invention. A further understanding of the nature and advantages of the present invention may be realized by reference to the remaining portions of the specification and the drawings, which forms a part of this disclosure.

### BRIEF DESCRIPTION OF THE DRAWINGS

The description and claims will be more fully understood with reference to the following figures and data graphs, which are presented as exemplary embodiments of the invention and should not be construed as a complete recitation of the scope of the invention.

FIG. 1 illustrates a retinal prosthetic in accordance with an embodiment of the invention.

FIG. 2 is a block diagram for an external controller in accordance with an embodiment of the invention.

FIG. 3 illustrates an algorithm for closed loop calibration in accordance with an embodiment of the invention.

FIG. 4 is a process for a closed loop calibration process in accordance with an embodiment of the invention.

### DETAILED DESCRIPTION

Retinal prosthetics are designed to restore visual function of the eye by direct stimulation of retinal ganglion cells (RGCs) via multi-electrode arrays, bypassing the photoreceptors and sending artificially generated signals directly to the brain. First-generation epiretinal implants restore crude visual sensations, but a high resolution, faithful representation of the visual scene has yet to be realized in these models. In first-generation epiretinal implants, each electrode simultaneously and indiscriminately activates many RGCs, creating a highly unnatural pattern of activity. Specifically, the human retina contains more than 20 different types of RGCs, each with a unique visual function and timing of light responses. Vision arises from spatial and temporal integration of these signals. Because the diverse RGC types are interleaved on the surface of the retina, coarse electrical stimulation inevitably activates multiple RGC types simultaneously. Thus, to reproduce high-fidelity vision using an epiretinal implant, the electrical stimulation must respect the diversity of RGC types by precisely controlling activation of neurons at the single-neuron scale. Ex vivo experiments on the primate retina have shown that this is often achievable with single electrode electrical stimulation, but not always.

Indeed, it remains difficult to selectively stimulate a specific individual RGC in isolation using a single electrode. A solution to this problem is using spatially patterned multi-electrode stimulation. However, such an approach is hampered by a combinatorial explosion of complexity. For example, one next generation implant being developed has 1024 electrodes each of which can deliver 64 current amplitudes, for a total of 64¹⁰²⁴ multi-electrode patterns, far too many to test exhaustively. Systems and methods described herein model the response of RGCs to multi-electrode stimulation, which can be used to calibrate retinal prosthetics. In various embodiments, the model can further be used to generate stimulation patterns that activate a cell more selectively. In numerous embodiments, the microelectrode array that delivers stimulation may also record cell responses, which in turn enables closed-loop calibration. An example retinal prosthetic that can utilize stimulation patterns is described in U.S. patent application Ser. No. 17/441,261, filed Mar. 23, 2020 and published as U.S. 2022/0168571, titled “Systems and Methods for Artificial Sight Prosthetics”, the entirety of which is incorporated by reference herein. Artificial sight prosthetics are discussed below to provide context, followed by a discussion of multi-electrode stimulation calibration methods.

### Artificial Sight Prosthetics

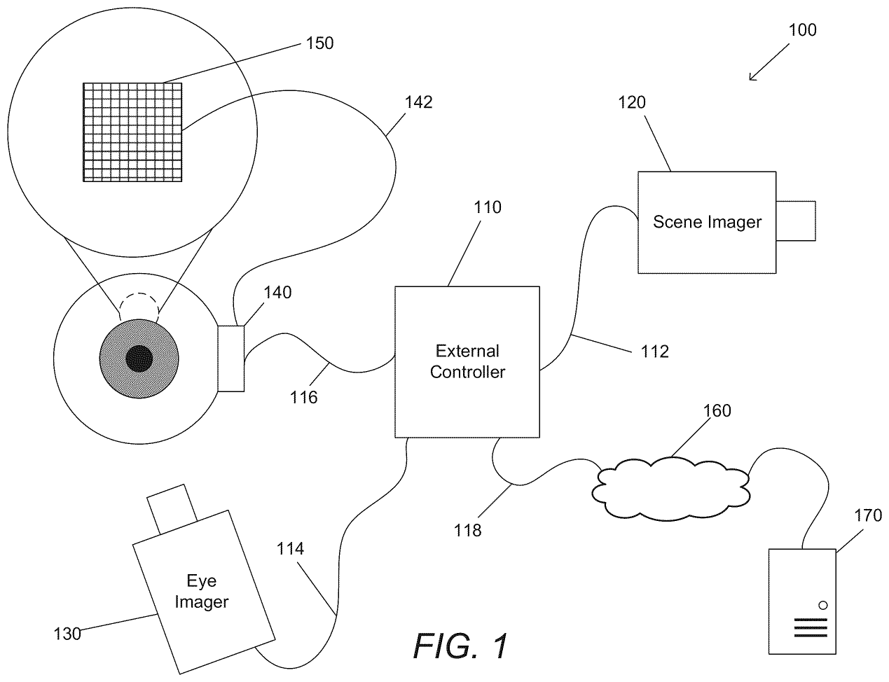

Turning now to FIG. 1, an example system for artificial sight as described in U.S. patent application Ser. No. 17/441,261 is illustrated. System 100 includes an external controller 110. In many embodiments, external controllers carry processing circuitry necessary to perform various artificial sight processes. In numerous embodiments, external controllers are capable of receiving image information describing a scene from a scene imager, eye position information from an eye imager. In many embodiments, the eye imager is capable of measuring and/or tracking the pupil and/or iris of a patient, fiduciary marks on a contact lens, blood vessels within the eye, the stimulation interface and/or the implanted controller in order to accurately measure the eye position information. External controllers can use image information and eye position information to compute field of view in a visual scene and compile dictionaries based on the received image information and eye position information. External controllers can transmit and receive data via at least one input/output interface. External controller input/output interfaces can transmit information across at least one communication channel. Channels can be either wired or wireless. External controllers are capable of utilizing at least two channels, where one channel is used to communicate with an input/output interface of an implanted controller, and where one channel is used to communicate with imagers. In some embodiments, external controllers are capable of communicating with configuration systems via a third channel. However, in some embodiments, communication to and from configuration systems can be accomplished via the same channel as used by the imagers. In a variety of embodiments, the external controller uses a wireless communication channel for exchanging data with the implanted controller, and a wired communication channel for exchanging data with the imagers. The wireless communication channel can be any number of different wireless channels, such as, but not limited to, radio communication (e.g. via the Bluetooth standard, or any other wireless communication method), magnetic communication methods (e.g. near-field magnetic induction communication), ultrasound communication, optical communication, and/or any other low power communication method that is safely usable in conjunction with an implanted controller adjacent to the human eye. In numerous embodiments, the external controller is further capable of transmitting power wirelessly to an implanted controller.

External controller 110 is connected to a scene imager 120 via a communication channel 112. As discussed above, communication channel 112 can be wired or wireless. Communication channel 112 is capable of relatively high-bandwidth communications to enable external controller 110 to receive images of the scene at a high frame rate of approximately 10-240 Hz. Scene imagers can be any image sensor capable of capturing images of an external environment. In numerous embodiments, the scene imager is a video camera. In a variety of embodiments, scene imagers are pointed in an outward facing direction from the patient such that the imager captures images of scenes in front of the patient. In many embodiments, additional sensors are used to augment scene imagers with alternate types of data. For example, GPS coordinates, LIDAR, alternate photosensors, sonar, and/or any other sensor configuration can be used as appropriate to the requirements of specific applications of embodiments of the invention. In some embodiments, scene imagers include at least two cameras located at a fixed distance apart in order to acquire depth information regarding the scene.

External controller 110 is also connected to an eye imager 130 via a communication channel 114. Communication channel 144 is similar to communication channel 112, and enables relatively high-bandwidth communication between the external controller and the eye imager. Eye imagers can be any image capture device capable of accurately recording the motion of an eye. In numerous embodiments, the eye imager is a video camera capable of recording sufficient data as to be used for motion tracking of the eye and/or the point of focus of the eye. This data is collectively described as eye position data, and can include raw image data to be processed by an external controller, and/or processed eye position data depending on the capabilities of the specific implementation of the eye imager architecture. In numerous embodiments, eye position data can further include pupil diameter, eye accommodative state, and/or focal depth. In many embodiments, eye imagers are capable of high-resolution tracking. However, in various embodiments, eye imagers are incapable of measuring small, micro-saccadic eye movements at high frequency. In this case, external controllers and/or eye imagers can measure saccade and add simulated micro-saccades which statistically approximate natural micro-saccadic eye movement. In numerous embodiments, both the scene imager and eye imager are implemented on the same platform as the external controller. For example, in some embodiments, a pair of glasses can be used to house an outward facing scene imager, an inward facing eye imager, and the external controller, as well as any necessary cables and/or transmitters/receivers necessary to implement the communication channels. However, any number of different physical platform configurations can be utilized as appropriate to the requirements of specific applications of embodiments of the invention.

External controller 110 is connected to implanted controller 140 via a communication channel 116. In numerous embodiments, communication channel 116 is a relatively lower-bandwidth communication channel. Due to the low power requirements of the implanted controller, as well as because the implanted controller may be subject to movement due to natural biological movements (e.g. eye movements, muscle movement, etc.), it can sometimes be difficult to establish a high-bandwidth connection that has reliably low latency. In a variety of embodiments, communication channel 116 is implemented using a low-power Bluetooth connection. In numerous embodiments, communication channel 116 is implemented using a near field communication channel, such as, but not limited to, a near-field magnetic induction communication channel, and/or a radio frequency based communication channel and/or ultrasound channel.

Implanted controllers are capable of implementing numerous different artificial sight processes. In many embodiments, implanted controllers are implanted into a patient by mounting the implanted controller onto the exterior of the eye. In some embodiments, the implanted controller is implanted into the episcleral layer of the eye. In a variety of embodiments, the implanted controller is integrated into a contact lens or an intraocular lens. Implanted controllers can obtain dictionaries from external controllers as well as field of view information, and use the received data to continuously select stimulation pulses based on the dictionary. In numerous embodiments, implanted controllers are implemented as an application-specific integrated circuit (ASIC). However, implanted controllers can be implemented using field-programmable gate arrays (FGPAs), or as energy efficient, low-heat general purpose processing devices equipped with machine-readable instruction.

Implanted controller 140 is connected to stimulation interface 150 via a communication channel 142. Communication channel 142 is relatively high-bandwidth and can be implemented using wired or wireless communications methods. In some embodiments, power is transmitted across communication channel 142, or via an alternative power transmission channel. In many embodiments, stimulation interfaces include a dense grid of small electrodes that are surgically connected to the RGC layer of the retina. Where many retinal prosthetics use sparse grids (e.g. 60 electrodes) of large electrodes (e.g. 200 μm), dense electrode grids may have on the order of 1000 electrodes per square millimeter. In some embodiments, the electrodes are placed in a rectangular and/or hexagonal arrangement, where each electrode is between 8 and 15 micrometers in diameter, and each electrode is spaced between approximately 10-50 micrometers apart.

In a variety of embodiments, the electrodes may have diameters of 5-20 μm and spacing of 10-60 μm. In numerous embodiments, the grid is connected to the retina with a semi-regular microwire bundle of approximately the same density as the electrode grid itself. In many embodiments, an interposer device is used to “zoom in” the density of electrodes on the interface to a higher density.

In numerous embodiments, stimulation interfaces include recording circuitry enabling them to record the response of RGCs to electrical stimulation provided by the stimulation interface, and/or their spontaneous electrical activity. These recordings can be used to create the dictionary entries that specify the probability of firing of each RGC in response to a pattern of stimulation. To accomplish this, in some embodiments, the recording circuitry is capable of recording voltages with approximately 10 bits of resolution over a range on the order of hundreds of microvolts, with approximately 10 microvolts of front-end noise, and at a sampling rate of approximately 20 kHz. However, the sensitivity and capabilities of the recording circuitry can be modified, and/or any of a number of different electrode arrangements can be used as appropriate to the requirements of specific applications of embodiments of the invention. In numerous embodiments, a data-compressive sensor array.

Interfaces can selectively apply variable stimulation to any of the electrodes in the electrode array based on instructions received from the implanted controller. Stimulation pulses can be monophasic, biphasic, triphasic, or multiphasic in time, with amplitude from approximately 0.1-10 μA, and duration of approximately 25-100 μsec. In numerous embodiments, interfaces also are capable of recording electrical impulses from adjacent RGCs and transmit the information back to the implanted controller, which in turn can transmit the recorded responses to the external controller. In some embodiments, the implanted controller and the stimulation interface are implemented as part of the same piece of hardware, and therefore the implanted controller is inside the eye itself, rather than internal to the body but on the external face or side of the eye.

External controller 110 is further connected to a network 160 via a communication channel 118 that gives access to a configuration server 170. Communication channel 118 is any communication channel that can be used to access configuration server 170. For example, in numerous embodiments, communication channel 118 is a connection to the Internet, which enables the passage of configuration data from configuration server 170 to the external controller 110. However, any number of different network and communication channel infrastructures can be used to connect the external controller to the configuration server as appropriate to the requirements of specific applications of embodiments of the invention. Configuration servers are used to provide updates to external controllers, eye imagers, or scene imagers as needed. In some embodiments, updates can be provided to implantable controllers as well. Received updates can include, but are not limited to, pre-processed dictionaries, calibration information for any component, or any other information required by the system as appropriate to the requirements of specific applications of embodiments of the invention. In some embodiments, the configuration server generates an initial global dictionary which can be used to generate smaller, faster, and/or more personalized dictionaries. In a variety of embodiments, the configuration server obtains pre-processed dictionaries from other devices. In numerous embodiments, the configuration server utilizes electrical recording and stimulation data to generate dictionaries.

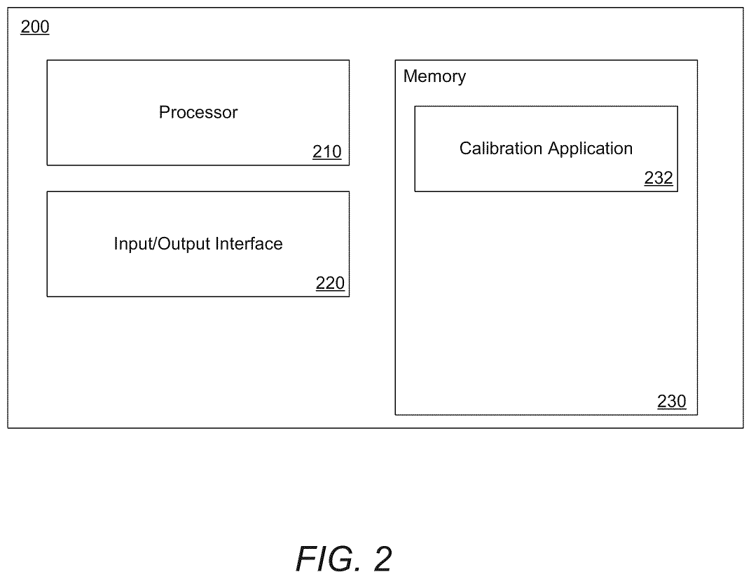

Turning now to FIG. 2, a block diagram for an external controller in accordance with an embodiment of the invention is illustrated. External controller 200 includes a processor 210. Processors can be any logic circuitry capable of executing logical processes including (but not limited to) central processing units (CPUs), graphics processing units (GPUs), application specific integrated circuits (ASICs), field programmable gate arrays (FPGAs), and/or any other logic circuitry as appropriate to the requirements of specific applications of embodiments of the invention. The controller 200 further includes an input/output (I/O) interface 220. I/O interfaces are capable of communicating with other devices via wired connections, wireless connections, and/or any combination thereof. The controller 200 additionally includes a memory 230. Memories can be made of volatile memory, nonvolatile memory, or a combination thereof. The memory 230 contains a calibration application capable of configuring the processor to perform various calibration processes as described herein. While a particular artificial sight prosthetic is discussed with respect to FIG. 1, as can be readily appreciated, different architectures may be calibrated. Further, some architectures may utilize internal controllers for calibration processes. Calibration methods for artificial sight prosthetics are discussed in further detail below.

### Calibration Methods

As noted above, an issue with single electrode retinal stimulation is selectivity. Multielectrode stimulation, however, increases the complexity of the stimulation regime. In order to calibrate a retinal prosthetic for multi-electrode stimulation, a generalizable model of neuron response is described which can be used to systematically and efficiently improve the selectivity of epiretinal stimulation over single electrode stimulation. As the described model has few parameters, efficient closed-loop calibration is possible, which in turn enables a smoother, less labor-intensive user experience.

First, a single-site (linear) model of extracellular stimulation is provided. Consider an applied stimulus current x∈ [symbol] ^{d}, where d is the number of stimulating electrodes. For a given activation site (a hypothesized region of the cell with a high density of voltage gated sodium channels), the probability pᵢ(x) of evoking a spike at site i is assumed to follow a sigmoidal function of the weighted sum of currents on the three electrodes: pᵢ(x)=σ(w_{i,0}+wᵢ^{T}x), where wᵢ∈ [symbol] ^{d} is a vector of weights on the multi-electrode currents, w_{i,0} is a scalar bias term for site i, and σ(x)=1/(1+exp (−x)). This formulation is consistent with known mechanisms of extracellular activation of neurons, as the extracellular voltage and activating function (proportional to the second spatial derivative of the voltage along the direction of the axon) are proportional to the stimulating current, for both the disk and point source electrode approximations. Under this interpretation, the quantity wᵢ^{T}x represents an underlying biophysical function which leads to activation of the neuron, and −w_{i,0} is the threshold value of the function corresponding to probability level of pᵢ(x)=0.5. The sum of these terms is then fed into a sigmoid nonlinearity to produce a probability that captures the stochastic nature of extracellular stimulation.

Although each site i in the model responds linearly to applied current on several electrodes, the model also assumes that there are several activation sites on a given cell, any of which may lead to an action potential if sufficiently stimulated. Under the assumption that each site is activated independently of other sites, a multi-site model for multi-electrode stimulation can be constructed such that the total activation probability for the neuron is the probability that any of its activation sites is stimulated:

$$p ( x ) = 1 - {\prod }_{i = i}^{m} ( 1 - {p}_{i} ( x ) )$$

With m the number of activation sites on the cell accessible by the stimulating electrodes. The model parameters (wᵢ and w_{i,0}) are fitted with maximum likelihood estimation (MLE), to produce an estimate ŵ. This parametric model retains biophysical interpretability while remaining flexible enough to fit complicated activation profiles by7 varying the hyperparameter m. in order to determine the number of sites m, which is unknown a priori, an early stopping procedure can be performed: iteratively adding sites and measuring the McFadden pseudo-R² defined as

$${R}_{McF}^{2} = 1 - \frac{log {L}_{M}}{log {L}_{0}} ,$$

where L_{M} and L₀ are the likelihoods of the fitted model and the null model (which only includes an intercept), respectively, and terminating upon convergence. Models with different values of m can be quickly and efficiently fitted with MLE either sequentially or in parallel. Empirically, a value of m no greater than 6 is needed for cells stimulated with d=3 adjacent stimulating electrodes with 30 μm electrode pitch.

Calibration methods as described herein adaptively learn responses to all multi-electrode stimuli using a small number of stimuli. That is, the parameters w_{i,0} and wᵢ (and implicitly the hyperparameter number of sites m) are learned for each cell using a small number of stimulation trials. This can be framed as a problem in optimal experimental design and active learning, where the goal is to learn the underlying model that produces a data set while minimizing the number of measurements needed. The experimental design is considered optimal with respect to some statistical criterion which is typically a convex function of the Fisher information matrix. The inverse of the Fisher information matrix provides a lower bound on the variance of any unbiased estimator \hat{θ} of an underlying true parameter θ. The Fisher information for a single-parameter Bernoulli experiment with probability of success θ and T independent trials is given by:

$$I ( θ ) = \frac{T}{θ ( 1 - θ )} .$$

Consider a data set with N currently levels and empirically measured activation probabilities for a given RGC,{(xₖ, \tilde{p}ₖ)}ₖ₌₁^{N}. The true underlying multi-site model fit to this data is denoted as wₜᵣᵤₑ. The predicted activation probabilities for the neuron at each current level k using the model fit are then pₖ(wₜᵣᵤₑ). Treating each current level as a different Bernoulli experiment with Tₖ independent trials, the Fisher information at each current level is written as:

$${I}_{pk} ( {p}_{k} ( {w}_{true} ) ) = \frac{{T}_{k}}{{p}_{k} ( {w}_{true} ) ( 1 - {p}_{k} ( {w}_{true} ) )}$$

In matrix form for all current levels, the N×N Fisher information is written as:

$${I}_{p} ( p ( {w}_{true} ) ) = diag ( {\{ \frac{{T}_{k}}{{p}_{k} ( {w}_{true} ) ( 1 - {p}_{k} ( {w}_{true} ) )} \}}_{k = 1}^{N} )$$

Reparametrizing the Fisher information in terms of model parameters wₜᵣᵤₑ, with the total number of model parameters l≡m×(d+1), the l×l Fisher information matrix is:

$${I}_{{w}_{true}} ( {w}_{true} ) = {J ( {w}_{true} )}^{T} {I}_{p} ( p ( {w}_{true} ) ) J ( {w}_{true} )$$

where J(wₜᵣᵤₑ) is the N×l Jacobian matrix defined as

$${J}_{ij} = \frac{\partial {p}_{i}}{\partial {w}_{j}}$$

evaluated at ine true parameter wₜᵣᵤₑ.

With the Fisher information matrix for a data set, a statistical criterion of the information is chose to optimize with the experimental design. In A-optimized designs, the goal is to minimize the variance of estimation error, approximated by the trace of the inverse of the Fisher information. The variance of the estimator of p(wₜᵣᵤₑ) is given by var(p(wₜᵣᵤₑ))≈J(wₜᵣᵤₑ)I_{w}_{ₜᵣᵤₑ}(wₜᵣᵤₑ)⁻¹J(wₜᵣᵤₑ)^{T}.

In an experiment, the true parameter vector wₜᵣᵤₑ is unknown, and instead only the MLE fit of the multisite model, ŵ, is available to estimate the Fisher information. At each adaptive sampling step, the optimal allocation of trials Tₖ across current levels is chosen to minimize the variance of estimation error. Specifically, at each step of the closed-loop experiment,

$${T}^{*} = {arg min}_{T > 0} [ tr ( var ( p ( \hat{w} ) ) ) + λ \vert {‖ T ‖}_{1} - {T}_{budget} \vert ] ,$$

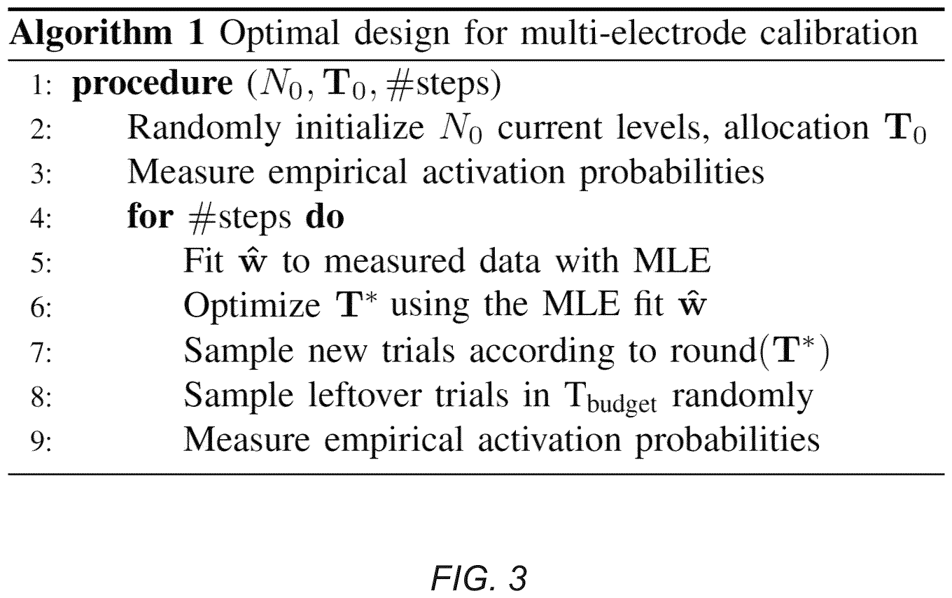

is optimized, where T_{budget} is the target number of new stimulation trials to perform on each adaptive step, and λ is an /1-regularization parameter. An entire closed loop procedure is described as an algorithm in accordance with an embodiment of the invention in FIG. 3.

In many embodiments, the location of each sensed RGC is determined, as well as its electrical image, i.e. the average electrical footprint of its spike. Empirical activation probabilities can be measured by stimulating at a sweep of current levels on each of the selected adjacent electrodes for stimulation, with the amplitude of the stimulating phase ranging between −1.8 μA to 1.8 μA. In numerous embodiments, the adjacent electrodes are three adjacent electrodes in the array and referred to as an electrode triplet. However, 2+ electrodes can easily be used, and three is not the only cardinality of the set of electrodes for multielectrode stimulation.

In many embodiments, the closed loop procedure is extended to simultaneously fit the multi-site model on multiple cells with the same set of electrodes and/or on several sets of electrodes by assuming the multi-site parameters in different cells and sets of electrodes to be independent. Experimentally, open loop scanning for a single electrode triplet takes approximately 30 minutes. The closed loop calibration method described herein reduces the time by 30× to approximately 1 minute. This is nontrivial because hundreds of electrode triplets would need to be scanned in order to complete a calibration, which would not be feasible in a clinical device operating under current open loop methods. Overall, this would cut the calibration time from several days to just a few hours. Once it is determined which electrodes can be used to selectively stimulate which neurons, then operation can proceed as normal by using said electrodes.

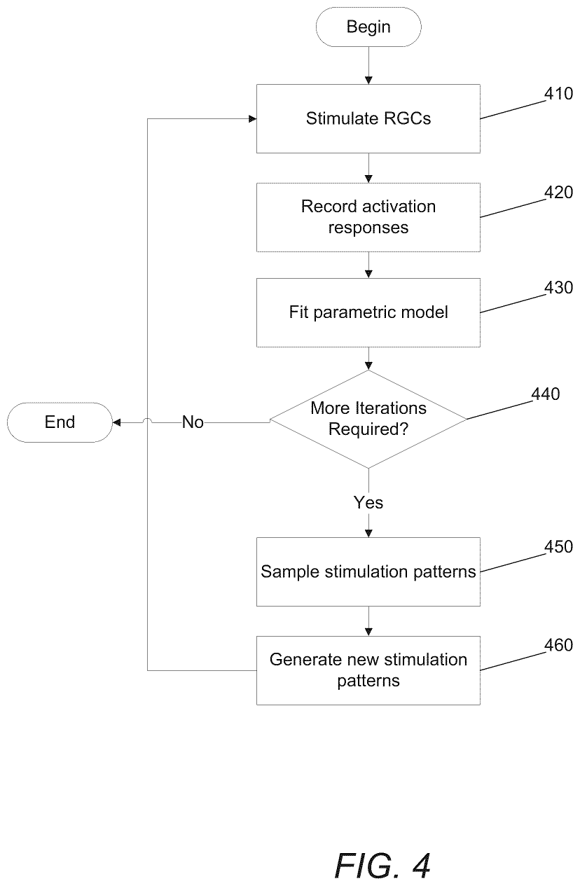

Turning to FIG. 4, a flow chart for a closed loop calibration method in accordance with an embodiment of the invention is illustrated. Process 400 includes stimulating (410) RGCs. In numerous embodiments, a random allocation of stimulation patterns (having random current and voltages) is used at initialization. Activation responses are recorded (420) and the parametric model is fitted (430) using the responses. If the stopping function has not been satisfied, thereby indicating more iterations are required (440), the stimulation patterns are sampled (450) and new patterns are generated (460) in accordance with the optimization of T*. The new patterns are tested via stimulation (410) and the activation responses are again recorded (420). The model can then be refit (430) with the new data, and the stopping function run again. Once no more iterations are required (440), the process concludes.

Indeed, although specific calibration methods are discussed above, any number of different modifications can be implemented in accordance with many different embodiments of the invention. It is therefore to be understood that the present invention may be practiced in ways other than specifically described, without departing from the scope and spirit of the present invention. Thus, embodiments of the present invention should be considered in all respects as illustrative and not restrictive. Accordingly, the scope of the invention should be determined not by the embodiments illustrated, but by the appended claims and their equivalents.

What is claimed is:

1. A method of calibrating a retinal prosthetic, comprising:
instantiating a parametric model for multi-electrode stimulation;
stimulating a plurality of retinal ganglion cells (RGCs) using a set of adjacent electrodes in a microelectrode array;
recording activation responses of the plurality of RGCs in response to the stimulation;
fitting the parametric model to the recorded activation responses;
determining a plurality of stimulation patterns for a next testing iteration using sequential measurement optimization, where each stimulation pattern comprises a current level and a voltage for each electrode in the set of adjacent microelectrodes;
stimulating the plurality of RGCs using a sampling of the determined plurality of stimulation patterns;
stimulating the plurality of RCGs using a different set of stimulation patterns;
recording new activation responses of the plurality of RGCs in response to the sampled stimulation patterns and the different set of stimulation patterns;
fitting the parametric model to the new activation responses; and
selecting a stimulation pattern that is most selective for a target RGC in the plurality of RGCs using the parametric model.

2. The calibration method of claim 1, where the sequential measurement optimization is A-optimal design.

3. The calibration method of claim 1, wherein recording activation responses comprises determining locations and an electrical image of each RGC in the plurality of RGCs.

4. The calibration method of claim 1, wherein the parametric model is pᵢ(x)=σ(w_{i,0}+wᵢ^{T}x), where wᵢ∈ [symbol] ^{d} is a vector of weights on multi-electrode currents, w_{i,0} is a scalar bias term for site i, and σ(x)=1/(1+exp (−x)).

5. The calibration method of claim 1, wherein a cardinality of the set of different stimulation patterns is determined by optimizing
$${T}^{*} = {arg min}_{T > 0} [ tr ( var ( p ( \hat{w} ) ) ) + λ \vert {‖ T ‖}_{1} - {T}_{budget} \vert ] ,$$
where T_{budget} is the cardinality, and λ is an /1-regularization parameter.

6. A retinal prosthetic, comprising:
a microelectrode array comprising a plurality of electrodes;
a processor; and
a memory, the memory containing a calibration application that configures the processor to:
instantiate a parametric model for multi-electrode stimulation;
stimulate a plurality of retinal ganglion cells (RGCs) using a set of adjacent electrodes in a microelectrode array;
record activation responses of the plurality of RGCs in response to the stimulation;
fit the parametric model to the recorded activation responses;
determine a plurality of stimulation patterns for a next testing iteration using sequential measurement optimization, where each stimulation pattern comprises a current level and a voltage for each electrode in the set of adjacent microelectrodes;
stimulate the plurality of RGCs using a sampling of the determined plurality of stimulation patterns;
stimulate the plurality of RCGs using a different set of stimulation patterns;
record new activation responses of the plurality of RGCs in response to the sampled stimulation patterns and the different set of stimulation patterns;
fit the parametric model to the new activation responses; and
select a stimulation pattern that is most selective for a target RGC in the plurality of RGCs using the parametric model.

7. The retinal prosthetic of claim 6, where the sequential measurement optimization is A-optimal design.

8. The retinal prosthetic of claim 6, wherein to record activation responses, the calibration application further configures the processor to determine locations and an electrical image of each RGC in the plurality of RGCs.

9. The retinal prosthetic of claim 6, wherein the parametric model is pᵢ(x)=σ(w_{i,0}+wᵢ^{T}x), where wᵢ∈ [symbol] ^{d} is a vector of weights on multi-electrode currents, w_{i,0} is a scalar bias term for site i, and σ(x)=1/(1+exp (−x)).

10. The retinal prosthetic of claim 6, wherein the calibration application further configures the processor to determine a cardinality of the set of different stimulation by optimizing
$${T}^{*} = {arg min}_{T > 0} [ tr ( var ( p ( \hat{w} ) ) ) + λ \vert {‖ T ‖}_{1} - {T}_{budget} \vert ] ,$$
where T_{budget} is the cardinality, and λ is an /1-regularization parameter.

11. A non-transitory machine readable medium containing program instructions that are executable by a set of one or more processors to perform a method comprising:
instantiating a parametric model for multi-electrode stimulation;
stimulating a plurality of retinal ganglion cells (RGCs) using a set of adjacent electrodes in a microelectrode array;
recording activation responses of the plurality of RGCs in response to the stimulation;
fitting the parametric model to the recorded activation responses;
determining a plurality of stimulation patterns for a next testing iteration using sequential measurement optimization, where each stimulation pattern comprises a current level and a voltage for each electrode in the set of adjacent microelectrodes;
stimulating the plurality of RGCs using a sampling of the determined plurality of stimulation patterns;
stimulating the plurality of RCGs using a different set of stimulation patterns;
recording new activation responses of the plurality of RGCs in response to the sampled stimulation patterns and the different set of stimulation patterns;
fitting the parametric model to the new activation responses; and
selecting a stimulation pattern that is most selective for a target RGC in the plurality of RGCs using the parametric model.

12. A non-transitory machine readable medium of claim 11, where the sequential measurement optimization is A-optimal design.

13. A non-transitory machine readable medium of claim 11, wherein recording activation responses comprises determining locations and an electrical image of each RGC in the plurality of RGCs.

14. A non-transitory machine readable medium of claim 11, wherein the parametric model is pᵢ(x)=σ(w_{i,0}+wᵢ^{T}x), where wᵢ∈ [symbol] ^{d} is a vector of weights on multi-electrode currents, w_{i,0} is a scalar bias term for site i, and σ(x)=1/(1+exp (−x)).

15. A non-transitory machine readable medium of claim 11, wherein a cardinality of the set of different stimulation patterns is determined by optimizing
$${T}^{*} = {arg min}_{T > 0} [ tr ( var ( p ( \hat{w} ) ) ) + λ \vert {‖ T ‖}_{1} - {T}_{budget} \vert ] ,$$
where T_{budget} is the cardinality, and λ is an /1-regularization parameter.
